(19) United States
(12) Patent Application Publication
(10) Pub. No.: US 2012/0226364 A1
(43) Pub. Date: Sep. 6, 2012

(54) METHOD FOR CONTROLLING AN ORTHOTIC OR PROSTHETIC JOINT OF A LOWER EXTREMITY

(75) Inventors: Philipp Kampas, Wien (AT); Martin Seyr, Wien (AT); Herman Boiten, Gottingen (DE); Sven Kaltenborn, Duderstadt (DE)

(73) Assignee: OTTO BOCK HEALTHCARE PRODUCTS GMBH, Wien (AT)

(21) Appl. No.: 13/508,518

(22) PCT Filed: Nov. 12, 2010
(86) PCT No.: PCT/EP2010/006896

§ 371 Date: May 7, 2012

(30) Foreign Application Priority Data
Nov. 13, 2009 (DE) 102009052887.3

(51) Int. Cl. A61F 2/48

(52) U.S. Cl. 623/24

(57) ABSTRACT

The invention relates to a method for controlling an orthotic or prosthetic joint of a lower extremity with a resistance device to which at least one actuator is associated, via which the bending and/or stretching resistance is changed depending on sensor data. During the use of the joint, status information is provided via the sensors. The sensor data are determined by at least one device for detecting at least two moments or a moment and a force. The sensor data of at least two determined values are linked by means of a mathematical operation and at least one auxiliary variable is thus calculated, on which the control of the bending and/or stretching resistance is based.

The invention relates to a method for controlling an orthotic or prosthetic joint of a lower extremity with a resistance device, which is assigned at least one actuator by way of which the bending and/or stretching resistance is changed in dependence on sensor data, information pertaining to the state being provided by way of the sensors during the use of the joint.

Knee joints for orthoses or prostheses have an upper connection part and a lower connection part, which are connected to each other by way of a joint device. Receptacles for an upper leg stump or an upper leg rail are generally arranged on the upper connection part, while a lower leg shaft or a lower leg rail is arranged on the lower connection part. In the simplest case, the upper connection part and the lower connection part are connected to each other pivotably by a single-axis joint. Only in exceptional cases is such an arrangement sufficient for ensuring the desired success, either support in the case of the use of an orthesis or a natural gait pattern in the case of use in a prosthesis.

In order to represent as naturally as possible or be conducive to the various requirements during the various phases of a step, or in the case of other tasks, resistance devices which offer a flexion resistance and an extension resistance are provided. The flexion resistance is used to set how easily the lower leg shaft or the lower leg rail swings backward in relation to the upper leg shaft or the upper leg rail when a force is applied. The extension resistance retards the forward movement of the lower leg shaft or the lower leg rail and forms, inter alia, a stretching stop.

The prior art, for example DE 10 2008 008 284 A1, discloses an orthopedic knee joint with an upper part and a lower part arranged pivotably thereon and assigned a number of sensors, for example a bending angle sensor, an acceleration sensor, an inclination sensor and/or a force sensor. The extension stop is determined in dependence on the sensor data.

DE 10 2006 021 802 A1 describes a control of a passive prosthetic knee joint with adjustable damping in the direction of flexion for the adaptation of a prosthetic device with upper connecting means and a connecting element to an artificial foot. The adaptation is for climbing stairs, a low-torque lift of the prosthetic foot being detected and the flexion damping being lowered in a lifting phase to below a level that is suitable for walking on level ground. The flexion damping may be raised in dependence on the changing of the knee angle and in dependence on the axial force acting on the lower leg.

The aim of the invention is to provide a method for controlling an artificial knee joint with which a situation-dependent adaptation of the flexion resistance and of the extension resistance is made possible. This object is achieved according to the invention by a method with the features of claim 1. Advantageous configurations and developments of the invention are presented in the dependent claims.

The method according to the invention for controlling an orthotic or prosthetic joint of a lower extremity with a resistance device, which is assigned at least one actuator by way of which the bending and/or stretching resistance is changed in dependence on sensor data, information pertaining to the state being provided by way of the sensors during the use of the knee joint, provides that the sensor data are determined by at least one device for detecting at least

two torques, or

one torque and one force, or

two torques and one force, or

two forces and one torque

and the sensor data of at least two of the variables determined are linked to one another by a mathematical operation and, as a result, at least one auxiliary variable is calculated and used as a basis for controlling the bending and/or stretching resistance. The sensors, which may be formed for example as knee or ankle torque sensors or axial load sensors, provide basic data, from which an auxiliary variable is calculated by way of a mathematical operation, for example addition, multiplication, subtraction or division. This auxiliary variable is sufficiently meaningful to be used as a basis for calculating an adaptation of the resistances. The auxiliary variable makes it possible rapidly and without great computational effort to provide a characteristic that can be used to calculate the current resistance to be set as a target variable and correspondingly activate the actuator to achieve the desired resistance. Provided in this case as the auxiliary variable are average torques, stress resultants, forces or distances, it being possible to determine as the auxiliary variable, for example, forces and torques that act at points of the orthesis or prosthesis that are not directly accessible by way of sensors. While the sensors only determine the forces or torques acting directly, calculation of the auxiliary variable can be used to obtain a variable for assessing the setting of the resistances that does not have to be detected directly. This broadens the possibilities for assessing which resistance should be set when, in which state of the movement or in which position of the joint or the prosthesis. In principle, it is possible to determine a number of auxiliary variables simultaneously and use them for control.

The sensors are arranged, for example, on the lower leg shaft or the lower leg rail and in the region of the joints. The auxiliary variable may represent a physical variable in the form of a virtual sensor. Since it is calculated, inter alia, from torques, forces and geometrical dimensions of the artificial joint, a force, a distance of a force from a reference point or a reference height, an average torque or a stress resultant at a reference height may be determined as the auxiliary variable. The distance of a force vector from an axis at a reference height, an average torque at a reference height or a stress resultant may be determined as the auxiliary variable. Thus, for example, the distance of the ground reaction force vector may be calculated by dividing a torque by the axial force. For this purpose, it is provided for example that the at least one device for detecting a torque, for example a torque sensor, detects a knee torque, so that the distance of the force vector of the ground reaction force for example at knee height, that is to say at the height of the knee joint axis, is determined as the auxiliary variable. It is also possible to determine the distance from a longitudinal axis, for example to determine the distance from a reference point on a longitudinal axis, the longitudinal axis connecting the devices for detecting the torques. Thus, for example, the distance of a force vector from the longitudinal axis of the lower connection part at the knee joint, that is to say the lower leg part, may be used. The distance of the force vector from an axis of a joint connection part in a reference position may be determined as the auxiliary variable by linking the data of at least one device for detecting two torques and one force.

In principle, it is also possible to use other reference heights, by the device for detecting a torque being fitted at the height of the reference height or by the torque at a reference height being calculated by weighted addition of two torques that are not located at the reference height. An average torque or a stress resultant may be determined as the auxiliary variable by a component at a reference height. The auxiliary variable that is detected with the virtual sensor, that is to say by mathematical linking of a number of sensor values, is calculated in a computing unit, for example in a microprocessor.

Specifically, the following variables may be emphasized as auxiliary variables for controlling an artificial knee joint, that is to say the distance of the ground reaction force from the knee joint axis or the torque of the ground reaction force about the knee axis, the distance of the ground reaction force at the height of the foot or the torque that the ground reaction force produces about the lower leg axis at the height of the foot, in particular at the height of the floor.

A further possibility for calculating the auxiliary variable is that the distance of the force vector from the lower leg axis in a reference position is determined by the linking of data of two devices for detecting a torque and an axial force sensor. When reference is made to a torque sensor, this wording also includes devices for detecting a torque that are made up of a number of components and do not necessarily have to be arranged at the location at which the torque acts.

It is also possible that an average torque at a reference height is determined by a weighted addition or subtraction of the values of an ankle torque sensor and a knee torque sensor. The average torque is then the auxiliary variable on the basis of which the control is correspondingly set.

Furthermore, it is possible and provided that a transverse force exerted on a lower connection part, for example the foot, is determined as the auxiliary variable from the quotient of the difference between two torques, for example a knee torque and an ankle torque, and the distance between the torque sensors. On the basis of the determined auxiliary variable or number of auxiliary variables, the corresponding resistance value is then calculated and set. After the maximum for the auxiliary variable is exceeded, the resistance may be continuously lowered with the auxiliary variable, in order to make easier swinging through of the joint possible on ramps or stairs.

When a predetermined value for the auxiliary variable is reached or exceeded, the resistance device may be switched into the swing phase state, thereby obtaining a basic setting of the flexion damping and extension damping that is changed in comparison with the standing phase state. Suitable for this is the average torque or the distance of the ground reaction force vector at the height of the foot.

It is provided that sensors for determining the knee angle, a knee angle velocity, an upper leg rail position or an upper leg shaft position, a lower leg position or a lower leg shaft position, the changing of these positions and/or the acceleration of the orthesis or prosthesis are present and that the data thereof are also used, along with using the auxiliary variable, for controlling the resistance or the resistances.

In order that there is as smooth as possible an adaptation of the resistance to the conditions pertaining to the state, it is provided that not only the data acquisition and the calculation of the auxiliary variable but also the resistance adaptation take place in real time. The changing of the resistance preferably takes place continuously with the auxiliary variable and/or the sensor data, in order to perform a smooth adaptation of the change in control, so that the user of an orthesis or prosthesis is not confronted with abrupt changes in the behavior of the orthesis or prosthesis.

It is also provided that, when there is an established alleviation, that is to say reduction, of the ground reaction force on the orthesis or prosthesis, for example when the leg is raised, the flexion resistance is reduced and, when there is increasing loading, the flexion resistance is increased. In the case of such a standing function, which is latently present and always performed when the natural movement pattern occurs, the resistance may lead to a locking of the joint. The increasing and reducing of the resistance preferably take place continuously and make a smooth transition possible, approximating to a natural movement and leading to a secure feeling for the wearer of the prosthesis or orthesis. If the auxiliary variable changes, the lock or the increasing of the resistance that has been activated in the standing function can be canceled or reduced, for example on the basis of the changing of the spatial position of the prosthesis or orthesis.

In principle, it is provided that the transition from the standing phase into the swing phase takes place load-dependently; it is likewise possible to move smoothly from the resistance setting for the standing phase into the resistance setting for the swing phase by gradual adaptation of the resistances and, if need be, that is to say when corresponding data for the auxiliary variable are present, to return similarly gradually into the standing phase again. This is advantageous in particular to make a swing phase on the ramp possible, by using the transverse force in the lower leg as the auxiliary variable.

A further aspect of the invention provides that the resistance is changed in dependence on a measured temperature. This makes it possible to protect the resistance device or other components of the artificial orthotic or prosthetic joint from excessive heating. Heating can even cause the joint to fail, because parts of the joint lose their shape or structural strength or because the electronics are operated outside the allowed operating parameters. The resistance is in this case preferably changed such that the dissipated energy is reduced. On account of the lower amount of energy to be converted, the resistance device or other components of the artificial joint can cool down and operate in a temperature range for which they are intended. In addition, it may be provided that the resistance device is adapted such that changes that occur on account of a change in temperature are balanced out. If, for example, the viscosity of a hydraulic fluid is reduced as a result of the heating, the resistance device may be correspondingly adjusted to continue to offer the accustomed flexion resistances and extension resistances, in order that the user of the prosthesis or orthesis can continue to rely on a familiar behavior of the artificial joint.

In a variant it is provided that the resistance is increased for the standing phase, for example during walking, when the temperature is increasing. In this case, both the extension resistance and the flexion resistance may be increased. The increased resistance has the effect that the user is forced to walk more slowly and consequently can introduce less energy into the joint. As a result, the joint can cool down, so that it can operate within the permissible operating parameters.

A further variant provides that, when walking, the bending resistance is reduced for the swing phase when the temperature is increasing. If the bending resistance is reduced in the, or for the, swing phase, this has the effect that the joint swings out further. The prosthetic foot consequently arrives forward for the heel strike later, whereby the user is in turn forced to walk more slowly, which leads to a reduced conversion of energy into heat.

The resistance may be changed when a temperature threshold value is reached or exceeded. The resistance may in this case be changed abruptly when a temperature threshold is reached or exceeded, so that a switching over of the resistance value or resistance values takes place. It is advantageously provided that a continuous changing of the resistance with the temperature takes place once the temperature threshold value is reached. How high the temperature threshold value is set depends on the respective structural parameters, materials used and the aimed-for uniformity of the resistance behavior of the prosthesis or orthesis. Inter alia, the resistance must not be increased in the standing phase to such an extent as to create a situation that is critical in terms of safety, for example when going down stairs.

The temperature-induced change in resistance is not the only control parameter of a change in resistance; rather, it is provided that such a temperature-induced change in resistance is superposed with a functional change in resistance. An artificial joint, for example a knee joint or ankle joint, is controlled situation-dependently by way of a large number of parameters, so that so-called functional changes in resistance, which take place for example on the basis of the walking speed, the walking situation or the like, are supplemented by the change in resistance on account of the temperature.

It may also be provided that, when a temperature threshold value is reached or exceeded, a warning signal is output to make the user of the prosthesis or orthesis aware that the joint or the resistance device is in a critical temperature range. The warning signal may be output as a tactile, optical or acoustic warning signal. Likewise, combinations of the various output possibilities are provided.

The temperature of the resistance device is preferably measured and used as a basis for the control; as an alternative to this, other devices may also be subjected to temperature measurement if they have a temperature-critical behavior. If, for example, control electronics are particularly temperature-sensitive, it is recommendable to monitor these electronics as an alternative or in addition to the resistance device and provide a corresponding temperature sensor there. If individual components are temperature-sensitive, for example on account of the materials used, it is recommendable to provide a measuring device at the corresponding points in order to be able to obtain corresponding temperature signals.

A setting device by way of which the degree of the change in resistance is changed may be provided. For example, it may be detected on the basis of determined data, for example the weight of the user of the prosthesis or orthesis or the determined axial force when stepping, that a disproportionately high change in resistance must take place. There is likewise the possibility that a manual setting device is provided, used for adapting the respective change in resistance, so that a change in resistance with a tendency to become greater or less in dependence on set or determined data can take place.

A device for carrying out the method as it is described above provides that a settable resistance device, which is arranged between two components of an artificial orthotic or prosthetic joint that are arranged one against the other in a jointed manner, and with a control device and sensors that detect information pertaining to the state in the device, is present. A setting device by way of which a change in resistance can be activated and/or can be deactivated is provided. This makes it possible, for example, to perform an optionally temperature-controlled change in resistance and deliberately activate or deactivate particular modes, a function or additional function, for example, of a knee control method.

A development of the invention provides that the bending and/or stretching resistance during the swing and/or standing phase or during standing is adapted on the basis of sensor data. While it is known from the prior art to retain a setting value once reached for the swing or standing phase until a new gait phase occurs, it is provided according to the invention that an adaptation of the flexion and/or extension resistance is variably set during the standing and/or swing phase. Thus, during the standing phase or the swing phase, a continuous adaptation of the resistance takes place when there are changing states, for example increasing forces, accelerations or torques. Instead of setting the flexion resistance and extension resistance by way of switching thresholds which, once reached, form the basis for the setting of the respective resistances, it is provided according to the invention that a variable, adapted setting of the resistances takes place, for example on the basis of an evaluation of characteristic diagrams. It is provided that a characteristic diagram for the flexion resistance in relation to the knee lever and the knee angle is set up and the control of the resistance takes place on the basis of the characteristic diagram.

In order to control artificial joints on the basis of sensor data, those sensors that are specifically necessary to ensure a safety standard in the detection of gait phase transitions are arranged. If sensors that go beyond the minimum required are used, for example to raise the safety standard, this redundancy of sensors makes it possible to realize controls that do not use all of the sensors arranged in or on the joint and nevertheless maintain a minimum standard of safety. It is provided that the redundancy of the sensors is used to realize alternative controls which, in the case of a failure of sensors, still make walking with a swing phase possible with the sensors that are still operating, and offer a minimum standard of safety.

Furthermore, it may be provided that the distance of the ground reaction force vector from a joint part is determined and the resistance is reduced whenever a threshold value of the distance is exceeded, that is to say whenever the distance of the ground reaction force vector lies above a minimum distance from a joint part, for example from a point on the longitudinal axis of the lower leg part at a specific height or from the pivot axis of the knee joint.

The flexion resistance may be reduced in the standing phase to a value suitable for the swing phase if, inter alia, an inertial angle of the lower leg part that is increasing in relation to the vertical is determined. The increasing inertial angle of the lower leg part indicates that the user of the prosthesis or user of the orthesis is in a forward movement, the distal end of the lower leg part being assumed as the hinge point. It is provided that the reduction only takes place whenever the increase in the inertial angle is above a threshold value. Furthermore, the resistance may be reduced if the movement of the lower leg part in relation to the upper leg part is not bending, that is to say is stretching or remains constant, which suggests a forward movement. Equally, the resistance may be reduced if there is a stretching knee torque.

It may be provided that the resistance is only reduced in the standing phase if the knee angle is less than 5°. This rules out the possibility of the joint being undesirably given clearance during the swing phase and with a bent knee.

The resistance may also be reduced when there is a bending knee torque to a value that is suitable for the swing phase if it has been determined that the knee torque has changed from stretching to bending. The reduction in this case takes place directly after the changing of the knee torque from stretching to bending.

Furthermore, it may be provided that, after a reduction, the resistance is increased again to the value in the standing phase if, within a fixed time after the reduction of the resistance, a threshold value for an inertial angle of a joint component, for an inertial angle velocity, for a ground reaction force, for a joint torque, for a joint angle or for a distance of a force vector from a joint component is not reached. To put it another way, the joint is set again to the standing phase state unless, within a fixed time after a change to the swing phase state, a swing phase is actually established. The basis for this is that the triggering of the swing phase has already taken place before the tip of the foot has left the ground, in order to make a prompt initiation of the swing phase possible. Should, however, the swing phase then not be initiated, as is the case for example when there is a circumduction movement, it is necessary to switch again to the safe standing phase resistance. Provided for this purpose is a timer, which checks whether within a specific time an expected value for one of the variables referred to above is present. The resistance remains reduced, that is to say the swing phase remains activated, if a joint angle increase is detected, that is to say if a swing phase is actually initiated. It is likewise possible that, after the threshold value is reached and clearance for the swing phase is given, the timer is only switched on when a second threshold value that is smaller than the first threshold value is fallen below.

The invention may also provide that the bending resistance is increased, or not reduced, in the standing phase if an inertial angle of a lower leg part that is decreasing in the direction of the vertical and a loading of the forefoot are determined. The coupling of the sensor variable of a decreasing inertial angle of a lower leg part in the direction of the vertical and the presence of a loading of the forefoot make it possible for walking backward to be reliably detected and no swing phase to be triggered, that is to say not to reduce the flexion resistance in order to avoid an unwanted bending of the knee joint if, when walking backward, the fitted leg is placed backward and set down. This makes it possible for the fitted leg to be loaded in the bending direction without buckling, so that it is possible for a patient fitted with a prosthesis or orthesis to walk backward without having to activate a special locking mechanism.

A development of the invention provides that the resistance is increased, or at least not reduced, if the inertial angle velocity of a joint part falls below a threshold value or, to put it another way, a swing phase with a lowering of the flexion resistance is initiated when the inertial angle velocity exceeds a predetermined threshold value. It is likewise possible that it is determined by way of the determination of the inertial angle of a joint part, in particular of the lower leg part, and the inertial angle velocity of a joint part, in particular of the lower leg part, that the user of the prosthesis or user of the orthesis is moving backward and needs a knee joint that is locked or greatly retarded against flexion. Accordingly, the resistance is increased if it is not yet sufficiently great.

Furthermore, it may be provided that the variation in the loading of the forefoot is determined and the resistance is increased, or not reduced, if, with a decreasing inertial angle of the lower leg part, the loading of the forefoot is reduced. While, in the case of a forward movement, after the heel strike the loading of the forefoot only increases when the lower leg part has been pivoted forward beyond the vertical, when walking backward the loading of the forefoot decreases when there is a decreasing inertial angle, so that in the presence of both states, that is a decreasing inertial angle and a decreasing loading of the forefoot, walking backward can be concluded. Accordingly, the resistance is then increased to that value that is provided for walking backward.

A further characteristic may be the knee torque, which is detected and serves as a basis for whether the resistance is increased, or not reduced. If a knee torque acting in the direction of flexion is determined, that is to say if the prosthetic foot has been set down and a flexion torque in the knee is detected, there is a situation in which walking backward must be assumed, so that then a flexion lock, that is to say an increase of the resistance to a value that does not make bending readily possible, is justified.

It may also be provided that the point at which a force acts on the foot is determined and the resistance is increased, or not reduced, if the point at which a force acts moves in the direction of the heel.

The inertial angle of the lower leg part may be determined directly by way of a sensor device which is arranged on the lower leg part or from the inertial angle of another connection part, for example the upper leg part, and a likewise determined joint angle. Since the joint angle between the upper leg part and the lower leg part may also be used for other control signals, the multiple arrangement of sensors and the multiple use of the signals provide a redundancy, so that, even in the event of failure of one sensor, the functionality of the prosthesis or orthesis continues to be preserved. A changing of the inertial angle of a joint part can be determined directly by way of a gyroscope or from the differentiation of an inertial angle signal of the joint part or from the inertial angle signal of a connection part and a joint angle.

An exemplary embodiment of the invention is described in more detail below.

In the drawing:

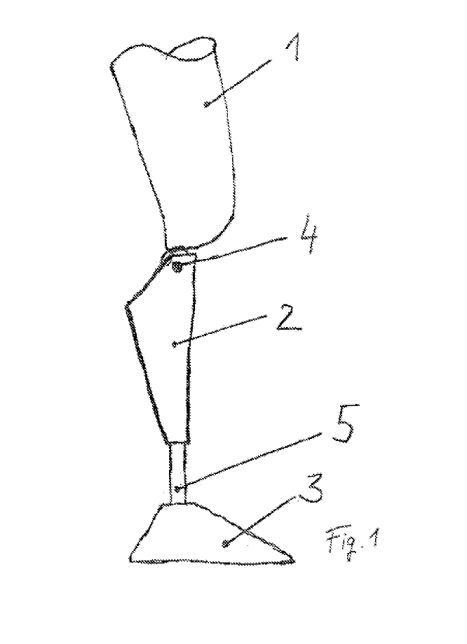

FIG. 1 shows a schematic representation of a prosthesis;

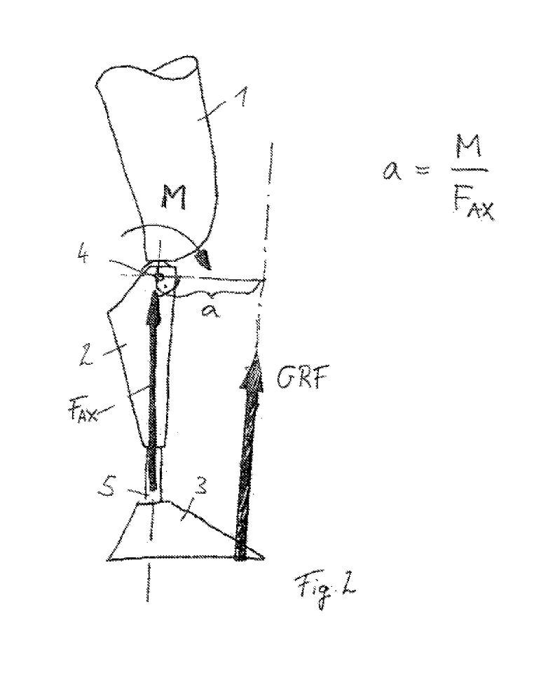

FIG. 2 shows a schematic representation for the calculation of a distance;

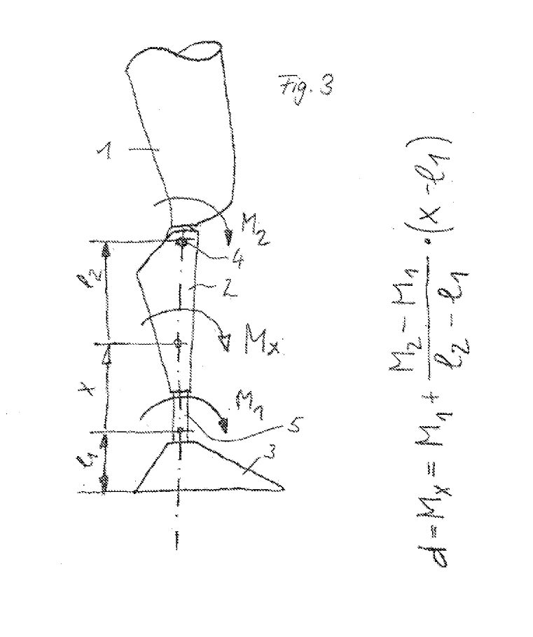

FIG. 3 shows a schematic representation for the calculation of an average torque;

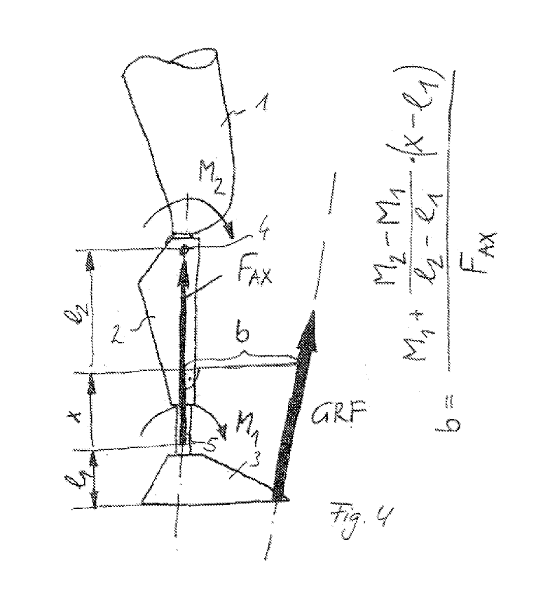

FIG. 4 shows a schematic representation for the calculation of a distance on the basis of a number of sensor values;

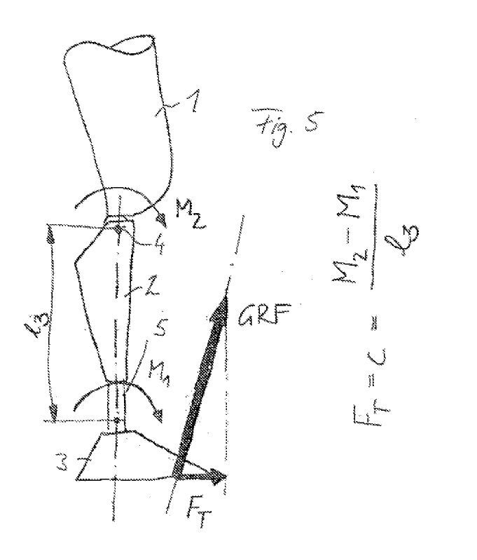

FIG. 5 shows a schematic representation for the calculation of a transverse force;

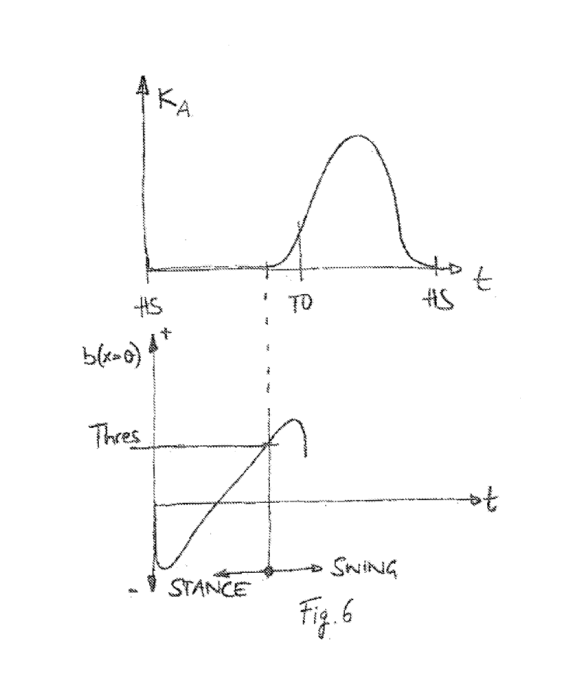

FIG. 6 shows representations of variations in values of the knee angle and an auxiliary variable over time;

FIG. 7 shows the behavior of characteristics when there is increasing resistance in the standing phase;

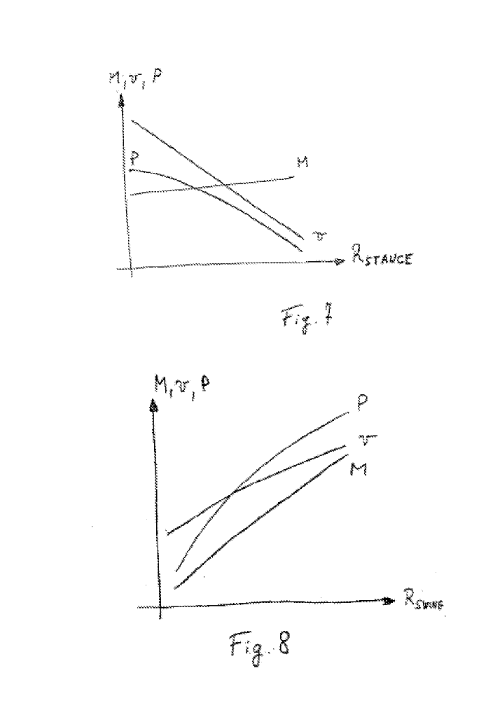

FIG. 8 shows the behavior of characteristics when there is increasing resistance in the swing phase;

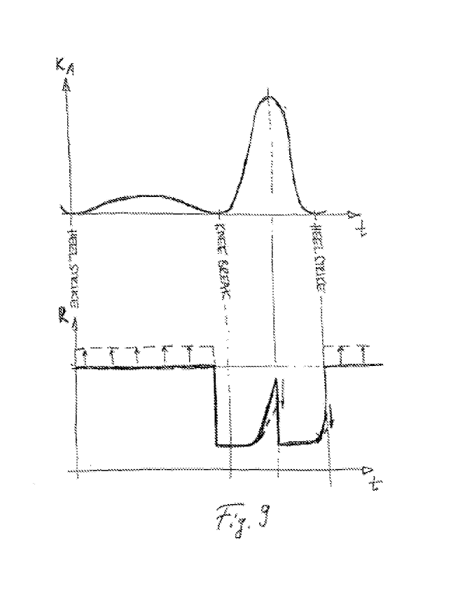

FIG. 9 shows a variation in the knee angle and a resistance curve when walking on level ground;

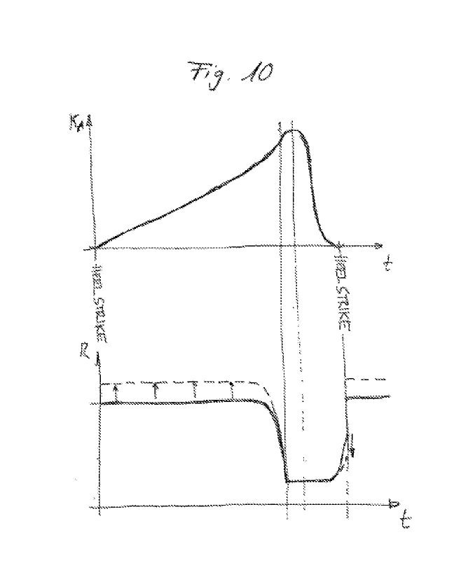

FIG. 10 shows a variation in the knee angle and a resistance curve when walking on an inclined level;

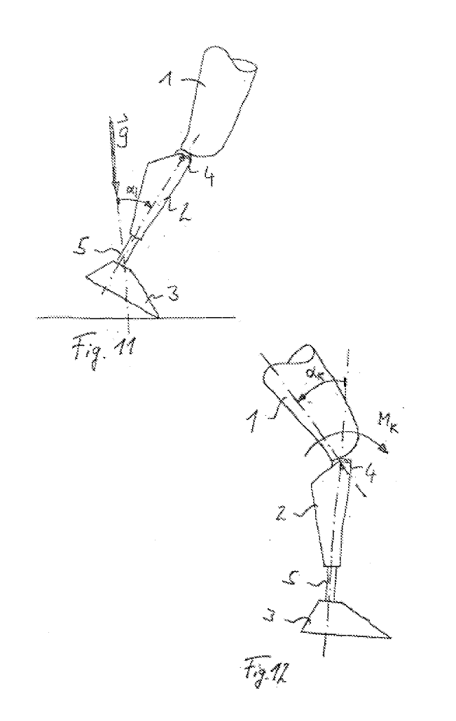

FIG. 11 shows a representation of the sign convention for the inertial angle and a schematic representation of a prothesis when walking backward;

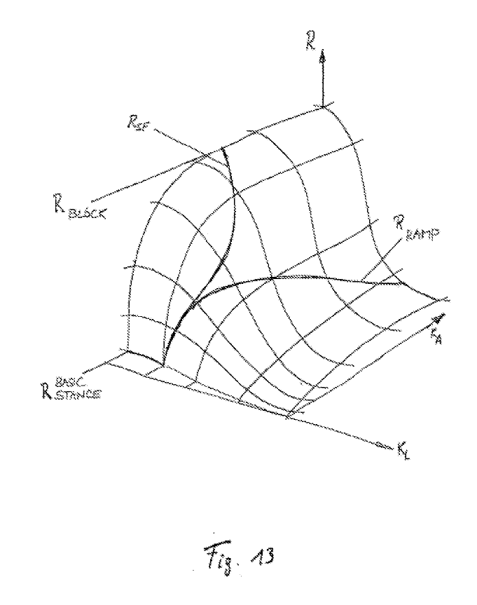

FIG. 12 shows a representation of the sign convention for the knee angle and the knee torque;

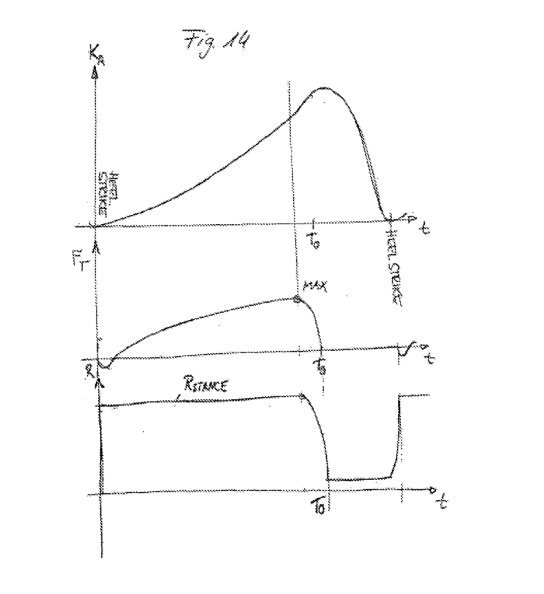

FIG. 13 shows a characteristic diagram for the resistance in relation to the knee angle and the knee lever;

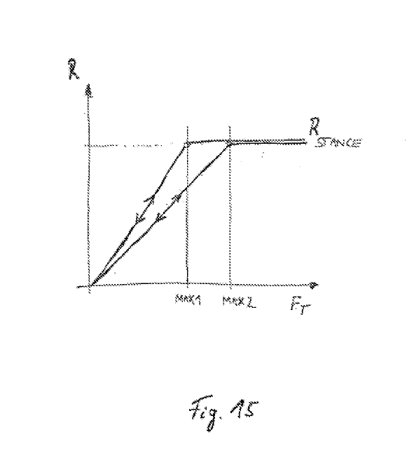

FIG. 14 shows characteristics when walking on inclined levels; and

FIG. 15 shows the resistance behavior for different transverse force maxima.

In FIG. 1, a schematic representation of a leg prosthesis with an upper leg shaft 1 for receiving an upper leg stump is shown. The upper leg shaft 1 is also referred to as the upper connection part. Arranged on the upper connection part 1 is a lower connection part 2 in the form of a lower leg shaft with a resistance device. Arranged on the lower connection part 2 is a prosthetic foot 3. The lower connection part 2 is pivotably fastened to the upper connection part 1 by way of a joint 4. Arranged in the joint 4 is a torque sensor, which determines the effective knee torque. Provided in the lower connection part 2 is a connecting part 5 to the prosthetic foot 3, in which a device for determining the effective axial force and the ankle torque is accommodated. Angle sensors and/or acceleration sensors may also be present. It is possible that not all the sensors are present in a leg prosthesis or additional sensors are present.

Apart from the resistance device, which offers the bending and stretching resistance, in the lower connection part 2 there is a control device, by way of which it is possible to change the respective resistance on the basis of the received sensor data and the evaluation of the sensor data. For this purpose, it is provided that the sensor data are used for producing at least one auxiliary variable, which is obtained by way of a mathematical linking of two or more sensor data. This makes it possible for a number of force or torque sensors to be linked to one another to calculate forces, distances and/or torques that are not acting directly in the region of the sensors. For example, it is possible to calculate stress resultants, average torques or distances in specific reference planes, in order on this basis to be able to assess which functions must be performed at the time in question in order that a gait pattern that is as natural as possible can be achieved. Referred to here as a function are those control sequences that occur in the course of a natural movement, whereas a mode is a switching state that is set by an arbitrary act, for example by actuating a separate switch or by a deliberate, possibly deliberately unnatural, sequence of movements.

In FIG. 2, it is schematically represented how the distance a of the ground reaction force vector GRF from the torque sensor is calculated as an auxiliary variable. The auxiliary variable a is in the present case the so-called knee lever, which is likewise represented in FIG. 13 and will be described in connection with a characteristic diagram control—though there with the opposite sign. The distance a is calculated from the quotient of the knee torque M and the axial force F_{AX}. The greater the knee torque M is in relation to the axial force F_{AX}, the greater the distance a of the ground reaction force vector GRF at the reference height, which in the present case forms the knee axis. On the basis of the auxiliary variable a, it is possible to vary the stretching resistance and/or the bending resistance, since this auxiliary variable a can be used to calculate whether and in which stage of the standing phase or swing phase the prosthesis is, so that on this basis a predetermined bending and/or stretching resistance is set. It can be determined by changing the auxiliary variable a how the movement at the time in question is proceeding, so that an adaptation of the stretching and/or bending resistance can take place within the movement, including within the standing phase or the swing phase. The changing of the resistances preferably takes place continuously and in dependence on the changing of the auxiliary variable or the auxiliary variables.

In FIG. 3, the auxiliary variable d is determined as an average torque Mₓ at the height x from the floor. In the example represented, the calculation takes place at the height of the foot, so that the value 0 can be assumed for the variable x. The average torque M, which is determined at the height x of the lower connection part 2, is calculated by the formula

$$d = Mx = M 1 + \frac{M 2 - M 1}{l 2 - l 1} * ( x - l 1 )$$

where M₁ is the torque in the connecting part 5, that is to say generally the ankle torque, the torque M₂ is the knee torque, the length l₁ is the distance of the ankle torque sensor from the floor, the length l₂ is the distance of the knee torque sensor from the floor and the length x is the reference height above the floor at which the average torque Mₓ is to be calculated. The calculation of the auxiliary variable d takes place here solely on the basis of the measurement of two torque sensors and the mathematical linking described above. The average torque Mₓ can be used to conclude the loading within the lower connection part 2, from which the loading within the lower connection part 2 or the connecting part 5 can be calculated. Depending on the magnitude and orientation of the average torque, various loading scenarios that require an adapted setting of the bending and/or stretching resistance are evident. On the basis of the effective average torque Mₓ at the respective instant, which is stored as auxiliary variable d in the control, the respectively necessary adjustment can be performed in real time in the resistance device in order to set the corresponding resistance.

In FIG. 4 it is shown how a further auxiliary variable b in the form of the distance of the ground reaction force vector GRF from an axis, in this case the connection of the two devices for detecting torques, at a reference height in relation to the axial force vector F_{AX} can be calculated. The auxiliary variable b is calculated from

$$b = \frac{M 1 + \frac{M 2 - M 1}{l 2 - l 1} * ( x - l 1 )}{FAX}$$

where M₁ is the effective torque in the connecting part 5, for example the ankle torque at the height l₁ from the floor, the torque M₂ is the knee torque at the height of the knee axis 4, which lies at a distance of l₂ from the floor. The variable x is the reference height from the floor, the force F_{AX} is the effective axial force within the connecting part 5 or in the lower connection part 2. By changing the auxiliary variable b, it is possible, as prescribed, to set the respective resistances and adjust them to the given changes continuously, both during the swing phase and during the standing phase. This makes it possible to activate various functions, which are automatically detected, for example a standing function that is used for example to prevent the knee joint from bending unwantedly. In the specific case, this auxiliary variable at the height x=0 is used for triggering the swing phase.

In the assessment for the triggering, not only the exceeding of the threshold value for the auxiliary variable b₍ₓ₋₀₎ can be used, but also the tendency. Thus, in the case of walking backward, a reversed variation in the auxiliary variable can be assumed, that is to say a migration of the point at which a force acts from the toe to the heel. In this case, no reduction of the resistance should take place.

FIG. 5 schematically shows how the transverse force or tangential force F_{T} can be calculated as a fourth auxiliary variable c and used for the knee controlling method. The tangential force F_{T}, and consequently also the auxiliary variable c, is obtained from the quotient of the difference between the knee torque M₂ and the ankle torque M₁ and the distance l₃ between the knee torque sensor and the ankle torque sensor.

$$c = Ft = \frac{M 2 - M 1}{l 3}$$

The auxiliary variable c can be used, for example, to lower the flexion resistance continuously with a falling auxiliary variable in the terminal standing phase when walking on inclined levels, in order to make easier swinging through of the joint possible.

In FIG. 6 it is shown by way of example how an auxiliary variable can be used to determine the triggering of the swing phase. In the upper graph, the knee angle K_{A} is plotted over time t, beginning with the heel strike HS and a substantially constant knee angle in the course of the standing phase, up until a bending of the knee shortly before the lifting off of the forefoot at the time TO. During the swing phase, the knee angle K_{A} then increases, until, after the bringing forward of the foot as far as the stretching stop, it is again at zero and the heel sets down once again.

Underneath the knee angle diagram, the value of the distance b of the ground reaction force vector from the lower leg axis according to FIG. 4 at the reference height x=0 is plotted over time t. As soon as the auxiliary variable b has reached a threshold value THRES, this is the triggering signal for the control to set the resistances such that they are suitable for the swing phase, for example by reducing the bending resistance to facilitate bending shortly before the forefoot leaves the floor. The reduction of the resistance can in this case take place continuously, not abruptly. It is likewise possible, if the auxiliary variable b changes again and takes an unforeseen path, that the resistances are correspondingly adapted, for example that the resistance is increased or the knee joint is even locked.

Apart from the described control of the functions by way of an auxiliary variable, it is possible to use a number of auxiliary variables for controlling the artificial joint, in order to obtain a more precise adaptation to the natural movement. In addition, further elements or parameters that are not directly attributable to the auxiliary variables may be used for controlling a prosthesis or orthesis.

In the diagram in FIG. 7, the dependence of the characteristics knee torque M, power P and velocity v is plotted by way of example against the resistance R_{STANCE} in the standing phase in the case of a prosthetic knee joint. Arranged here in the prosthetic knee joint are a resistance device and an actuator, by way of which the resistance that opposes the bending and/or stretching can be changed. Apart from a prosthesis, a correspondingly equipped orthesis may also be used, and other joint devices are likewise possible as the area of use, for example hip or foot joints. In the resistance device, the mechanical energy is generally converted into thermal energy, in order to retard the movement of a lower leg part in relation to an upper leg part, and the same correspondingly applies to other joints.

The temperature of the resistance device depends here on how great the power P that is applied during the standing phase is. The power P depends on the effective knee torque M and the velocity v with which the knee joint is bent. This velocity depends in turn on the resistance R_{STANCE} with which the respective movement is opposed in the standing phase by the resistance device (not represented). If, in the standing phase, the flexion resistance is increased after the heel strike and, as the sequence progresses further with a commencing extension movement, the extension resistance is increased, the movement velocity of the joint components in relation to one another is reduced, and consequently so too is the movement velocity of the resistance device. The reduction of the velocity v, which is stronger than the slight increase in the torque M, has the effect of reducing the power P during the standing phase, so that the energy to be converted decreases in a way corresponding to the reducing power P. Accordingly, with cooling remaining the same, the temperature of the resistance device, or that component that is being monitored with regard to its temperature, is reduced.

In FIG. 8, the correlation of the described characteristics to the resistance R_{SWING} in the swing phase is represented. With a reduction of the resistance R during the swing phase, the walking speed v, the knee torque M and consequently also the applied power P are reduced, so that the energy to be converted is reduced. Accordingly, the temperature of the resistance device is reduced when there is a decreasing swing phase resistance. A standing and/or swing phase control that is controlled by way of the temperature may take place in addition to the control by way of the auxiliary variables described above, or else separately from it.

FIG. 9 shows in the upper diagram the knee angle K_{A} over time t, beginning with the so-called “heel strike”, which is generally performed with a stretched knee joint. During the setting down of the foot, a small flexion of the knee joint takes place, known as the standing phase flexion, in order to mitigate the setting down of the foot and the heel strike. Once the foot has been set down completely, the knee joint is fully stretched, until the so-called “knee break”, at which the knee joint is bent in order to move the knee joint forward and to roll over the forefoot. Proceeding from the “knee break”, the knee angle K_{A} increases up to the maximum knee angle in the swing phase, to then, after the bringing forward of the bent leg and the prosthetic foot, go over into a stretched position again, to then again set down with the heel. This variation in the knee angle is typical for walking on level ground.

In the lower diagram, the resistance R is plotted over time, in a way corresponding to the corresponding knee angle. This diagram shows the effect of a changing of the resistance in the swing phase and the standing phase that has been carried out, for example, on account of a temperature-induced change in resistance. Whether an extension or flexion resistance is applied depends on the variation in the knee angle; with an increasing knee angle K_{A}, the flexion resistance is effective, with a decreasing knee angle, the extension resistance. After the “heel strike”, there is a relatively high flexion resistance, after the reversal in the movement there is a high extension resistance. At “knee break”, the resistance is reduced, in order to facilitate the bending and bringing forward of the knee. This makes walking easier. After the lowering of the resistance at the “knee break”, the resistance is kept at the low level over part of the swing phase, in order to facilitate a swinging backward of the prosthetic foot. In order not to allow the swinging movement to become excessive, the flexion resistance is increased before reaching the knee angle maximum and the extension resistance is reduced to the low level of the swing phase bending after reaching the knee angle maximum and the reversal in the movement. The reduction of the extension resistance is retained even over the extension movement in the swing phase, until shortly before the “heel strike”. Shortly before reaching full stretching, the resistance is once again increased, in order to avoid hard impact with the stretching stop. In order to obtain sufficient certainty that uncontrolled buckling does not occur when the prosthetic foot is set down, the flexion resistance is also at a high level.

If the flexion resistance is then increased, which is indicated by the dashed line, the knee angle velocity slows down, and consequently also the walking of the user of the prosthesis. After the “heel strike”, there follows only a comparatively small bending in the standing phase flexion and a slow stretching, so that less energy is dissipated. The raising of the flexion resistance before reaching the knee angle maximum takes place in a less pronounced way than in the case of the standard damping, which is indicated by the downwardly directed arrow. As a result, the lower leg swings out further, and consequently so does the prosthetic foot, so that there is a greater time period between the “heel strikes”. The reducing of the flexion resistance in the swing phase flexion also leads to a reduction of the walking speed.

At the end of the swing phase extension, that is to say shortly before stepping and the “heel strike”, the extension resistance is reduced in comparison with the standard level. It is therefore provided that the extension resistance is reduced, so that the lower leg part becomes stretched more quickly. In order to avoid hard impact when stretching, the user of the prosthesis will walk more slowly, so that the power P is reduced, and consequently so too is the energy to be dissipated. During the standing phase between the “heel strike” and the “knee break”, both the flexion resistance and the extension resistance may be increased, in order to slow down the slight bending and stretching movement in order thereby to reduce the walking speed.

In FIG. 10, the variation in the knee angle when walking on a ramp, here on a downward sloping ramp, is shown in the upper representation. After the “heel strike”, there is a continuous increasing of the knee angle K_{A}, up to the knee angle maximum, without a “knee break” taking place. The reason for this is that, when walking on a ramp, the knee does not reach full stretching. After reaching the knee angle maximum, a quick bringing forward of the knee and of the lower leg takes place up to full stretching, which is accompanied by a renewed “heel strike”. The flexion resistance thereby remains at a constantly high level over much of the progression, until it is then lowered in order to make further bending of the knee possible, and consequently lifting off of the prosthetic foot and swinging through. This swinging through takes place after reaching the minimum of the resistance up until reaching the knee angle maximum. The extension resistance is subsequently kept at a low level, until it is once again raised shortly before stepping.

If there are then increased temperatures in the resistance device, the resistances are increased in the standing phase, in order to ensure a slow walking speed and slow buckling. After reaching the maximum bending angle in the swing phase, the extension resistance is reduced during the bringing forward of the prosthetic foot in comparison with the normal function, which likewise leads to a reduction of the energy to be dissipated.

Apart from the customary movement situation, in which a patient moves forward, in the daily movement profile there are many other situations, which should be responded to with an adapted control.

In FIG. 11, the prosthesis is represented in a situation in which the swing phase is normally triggered in the case of walking forward. In this situation, the patient is still on the forefoot and would then like to bend the hip, so that the knee also bends. However, the patient also arrives in the same situation when walking backward. Starting from a standing situation, when walking backward the fitted leg, in the present case the prosthesis, is set backward, that is to say opposite to the normal viewing direction of a user of the prosthesis. This has the effect that the inertial angle α₁ of the lower leg part 2 initially increases in relation to the direction of gravitational force, which is indicated by the gravitational force vector g, until the prosthetic foot 3 is set down on the ground. The hip joint should be assumed here as the pivot point or hinge point for the movement and for determining the increasing inertial angle α₁. The longitudinal extent or longitudinal axis of the lower leg part 2 runs through the pivot axis of the prosthetic knee joint 4 and preferably likewise through a pivot axis of the ankle joint or else centrally through a connection point between the prosthetic foot 3 and the lower leg part 2. The inertial angle α₁ of the lower leg part 2 can be determined directly by a sensor system arranged on the lower leg part 2; as an alternative to this, it may be determined by way of a sensor system on the upper leg part 1 and a knee angle sensor, which detects the angle between the upper leg part 1 and the lower leg part 2.

For determining the inertial angle velocity, a gyroscope may be used directly, or the changing of the inertial angle a₁ over time is determined, and this can be determined in terms of the amount and the direction. If there is then a specific inertial angle α₁ and a specific inertial angle velocity ω̅₁, a swing phase is initiated if a specific threshold value for the inertial angle velocity ω̅₁ is exceeded. If there is a decreasing inertial angle α₁, and additionally also a loading of the forefoot, walking backward can be concluded, so that the flexion resistance is not reduced but is retained or increased, in order not to initiate a swing phase flexion.

In FIG. 12, the prosthesis is shown in a state in which it has been set down flat on the ground. The representation serves in particular for defining the knee torque and the knee angle and also the sign convention used. The knee angle α_{K} corresponds in this case to the angle between the upper leg part 1 and the lower part 2. A knee torque M_{K} is effective about the joint axis of the prosthetic knee joint 4. The triggering of the swing phase may be supplemented by further criteria, for example by the knee torque M_{K} having to be stretching, that is to say positive or zero, by the knee angle α_{K} being virtually zero, that is to say by the knee being stretched and/or by the knee angle velocity being zero or stretching.

An elegant way of taking various parameters and parameter relationships into consideration is given by the use of a characteristic diagram. As a difference from switching that is controlled purely on the basis of a threshold value, the characteristic diagram makes it possible to set resistances that are variable and adapted to variations or combinations of the variables of the characteristic diagram. The auxiliary variables that have been described above may also be used for this.

In FIG. 13, a characteristic diagram for controlling walking on level ground is represented, set up for determining the resistance R to be set. The characteristic diagram is set up between the resistance R, the knee angle K_{A} and the knee lever K_{L}. The knee lever K_{L} is the distance at right angles of the resulting ground reaction force from the knee axis and can be calculated by dividing the effective knee torque by the effective axial force, as described in FIG. 2. There, the knee lever was described as auxiliary variable a—though with the opposite sign. Assumed as the maximum value for the resistance R is that value at which the joint, in the present case the knee joint, cannot bend, or only very slowly, without destroying a component. If the knee lever K_{L}=−a tends toward zero after an initial increase, and the lower leg had been tilted significantly rearwardly, which is typical for walking on level ground, the flexion resistance R is increased from a base flexion resistance to a maximum standing phase bending angle of, for example, 15° or just below that with increasing knee angle up to the block resistance R_{BLOCK}. Such a curve is represented in FIG. 13 as the normal standing phase flexion curve R_{SF}. The resistance device therefore limits the bending under standing phase flexion when walking on level ground. If the knee lever K_{L} increases, however, the flexion resistance is increased less. This behavior corresponds for example to walking down a ramp or a slowing-down step and is depicted by R_{RAMP}. The characteristic diagram makes a continuous transition between walking on level ground and walking on a ramp possible. Since not a threshold value but a continuous characteristic diagram is used, a transition between walking on level ground and walking on a ramp is also possible in the advanced stage of the standing phase.

In FIG. 14, the characteristics knee angle K_{A}, tangential force F_{T} and flexion resistance R that are characteristic of when walking on inclined levels, in the present case when walking down a slope, are represented over time t. After the “heel strike”, the knee angle K_{A} increases continuously up to the point in time of lifting off of the foot T₀. After that, the knee angle K_{A} increases once again, in order in the swing phase to bring the lower leg part closer to the upper leg part, in order to be able to set the foot forward. After reaching the maximum knee angle K_{A}, the lower leg part is brought forward and the knee angle K_{A} is reduced to zero, so that the leg is again in the stretched state in which the heel is set down, so that a new stepping cycle can begin.

After the “heel strike”, the tangential force F_{T} or transverse force assumes a negative value, passes through zero after the full setting down of the foot and then increases to a maximum value shortly before the lifting off of the foot. After the lifting off of the foot at the point in time T₀, the transverse force F_{T} is zero, up until the renewed “heel strike”.

The variation in the flexion resistance R is virtually constant and very high up to the maximum of the transverse force F_{T}, in order to counteract the force acting in the direction of flexion when going down a slope, in order that the patient is relieved and does not have to use the retained side to compensate for the swing of the moved artificial knee. After reaching the transverse force maximum, which lies before the lifting off of the foot, the flexion resistance R is reduced continuously with the tangential force, in order to make facilitated bending of the knee joint possible. After the lifting off of the forefoot at the point in time T₀, the flexion resistance R has its minimum value, in order that the lower leg can easily swing again rearwardly. If the lower leg is brought forward, the extension resistance is effective, also depicted in this diagram for reasons of completeness. With a decreasing knee angle, the resistance R is formed as the extension resistance, which is increased to a maximum value shortly before reaching the renewed setting down, that is to say shortly before the renewed “heel strike”, in order to provide extension damping, in order that the knee joint is not moved undamped to the extension stop. The flexion resistance is increased to the high value, in order that the required effective flexion resistance can be provided directly after the “heel strike”.

In FIG. 15, the ratio between the resistance R to be set and various transverse force maxima is represented. The decrease in resistance has been normalized here to the transverse force maximum. This is intended to achieve the effect that the resistance is brought down from a high value to a low value, while the transverse force tends toward the value zero from a maximum. The reduction is consequently independent of the height of the maximum of the transverse force. It goes from the standing phase resistance to the minimum resistance, while the transverse force goes from the maximum to zero. Should the transverse force rise again, the resistance is again increased, that is to say the user of the prosthesis can again exert greater loading on the joint, should he discontinue the movement. Here, too, a continuous transition between easy swinging through and renewed loading is possible, without a discrete switching criterion being used.

1. A method for controlling an orthotic or prosthetic joint of a lower extremity with a resistance device, which is assigned at least one actuator by way of which the bending and/or stretching resistance is changed in dependence on sensor data, information pertaining to the state being provided by way of sensors during the use of the joint, characterized in that the sensor data are determined by at least one device for detecting at least
two torques, or
one torque and one force, or
two torques and one force, or
two forces and one torque
and the sensor data of at least two of the variables determined are linked to one another by a mathematical operation and, as a result, an auxiliary variable is calculated and used as a basis for controlling the bending and/or stretching resistance.

2. The method as claimed in claim 1, characterized in that the sensor data are added to one another, multiplied, subtracted from one another and/or divided.

3. The method as claimed in claim 1 or 2, characterized in that the distance of a force vector from an axis at a reference height, an average torque at a reference height or a stress resultant is determined as the auxiliary variable.

4. The method as claimed in one of the preceding claims, characterized in that the distance of the force vector of the ground reaction force from the device for detecting a torque is calculated as the auxiliary variable by dividing the torque by the force.

5. The method as claimed in claim 4, characterized in that the distance of the force vector from the joint axis is calculated by dividing the joint torque by the axial force.

6. The method as claimed in one of the preceding claims, characterized in that an ankle and/or knee torque sensor is used as the device for detecting a torque.

7. The method as claimed in one of the preceding claims, characterized in that the distance of the force vector from an axis of a joint connection part in a reference position is determined as the auxiliary variable by linking the data of at least one device for detecting two torques and one force.

8. The method as claimed in one of the preceding claims, characterized in that an average torque at a reference height is determined as the auxiliary variable by weighted addition or subtraction of the values of devices for detecting two torques, in particular an ankle torque sensor and a knee torque sensor.

9. The method as claimed in one of the preceding claims, characterized in that a transverse force exerted on a lower connection part is determined as the auxiliary variable from the quotient of the difference between two torques and the distance between the two devices for determining the torques.

10. The method as claimed in one of the preceding claims, characterized in that, when a predetermined value for the auxiliary variable is reached or exceeded, the resistance device is switched into a swing phase state.

11. The method as claimed in one of the preceding claims, characterized in that the flexion resistance is lowered if there is a decreasing value of the auxiliary variable.

12. The method as claimed in one of the preceding claims, characterized in that sensors for determining the knee angle, the knee angle velocity, an upper leg position, a lower leg position, the changing of these positions and/or the acceleration of the orthesis or prothesis are arranged on the orthesis or prosthesis and the data thereof are used for controlling the resistance.

13. The method as claimed in one of the preceding claims, characterized in that the data acquisition and calculation and also the change in resistance take place in real time.

14. The method as claimed in one of the preceding claims, characterized in that the change in resistance is carried out continuously.

15. The method as claimed in one of the preceding claims, characterized in that, when there is an established increasing of the auxiliary variable, the resistance is increased up to a locking of the joint.

16. The method as claimed in one of the preceding claims, characterized in that, when there is an established reduction of the ground reaction force on the orthesis or prosthesis, the resistance is reduced and, when there is an increasing ground reaction force, the resistance is increased up to a locking of the joint.

17. The method as claimed in claim 16, characterized in that the locking of the joint is canceled if the auxiliary variable changes.

18. The method as claimed in one of the preceding claims, characterized in that the resistance is reduced after the increase on the basis of a detected changing of the spatial position of the orthesis or prosthesis or as a result of a detected changing of the position of a force vector in relation to the orthesis or prosthesis.

19. The method as claimed in one of the preceding claims, characterized in that a temperature sensor is provided and in that the resistance is changed in dependence on at least one measured temperature signal.

20. The method as claimed in claim 19, characterized in that the resistance is increased during the standing phase when there is increasing temperature.

21. The method as claimed in claim 19 or 20, characterized in that the bending resistance is reduced during the swing phase when there is increasing temperature.

22. The method as claimed in one of claims 19 to 21, characterized in that the resistance is changed when a temperature threshold value is reached or exceeded.

23. The method as claimed in one of claims 19 to 22, characterized in that the resistance is changed continuously with the changing temperature.

24. The method as claimed in one of claims 19 to 23, characterized in that the temperature-induced change in resistance is superposed with a functional change in resistance.

25. The method as claimed in one of claims 19 to 24, characterized in that a warning signal is output when a temperature threshold value is reached or exceeded.

26. The method as claimed in one of claims 19 to 25, characterized in that the temperature of the resistance device is measured and used as a basis for the control.

27. The method as claimed in one of claims 19 to 26, characterized in that a setting device by way of which the degree of the change in resistance is changed is provided.

28. The method as claimed in one of the preceding claims, characterized in that a characteristic diagram of the flexion resistance, the knee lever and the knee angle is set up and the control of the resistance takes place on the basis of the characteristic diagram.

29. The method as claimed in one of the preceding claims, characterized in that, in the case of a failure of devices for detecting torques, forces and/or joint angles, alternative control algorithms on the basis of the remaining devices are used for changing the stretching and/or bending resistance.

30. The method as claimed in one of the preceding claims, characterized in that the distance of the ground reaction force vector from a joint part is determined and the resistance is reduced if a threshold value of the distance is exceeded.

31. The method as claimed in claim 30, characterized in that the resistance is reduced in the standing phase if the knee angle is less than 5°.

32. The method as claimed in claim 30 or 31, characterized in that the resistance is reduced in the standing phase if an inertial angle of the lower leg part that is increasing in relation to the vertical is determined.

33. The method as claimed in one of claims 30 to 32, characterized in that the resistance is reduced if the movement of the lower leg part in relation to the upper leg part is not bending.

34. The method as claimed in one of claims 30 to 33, characterized in that the resistance is reduced if there is a stretching knee torque.

35. The method as claimed in one of claims 30 to 34, characterized in that, after a reduction, the resistance is increased again to the value for the standing phase if, within a fixed time after the reduction of the resistance, a threshold value for an inertial angle of a joint component, for an inertial angle velocity, for a ground reaction force, for a joint torque, for a joint angle or for a distance of a force vector from a joint component is not reached.

36. The method as claimed in one of claims 30 to 35, characterized in that, after a reduction, the resistance is increased again to the value for the standing phase if, after the reduction of the resistance and reaching a threshold value for an inertial angle of a joint component, an inertial angle velocity, a ground reaction force, a joint torque, a joint angle or a distance of a force vector from a joint component after the reduction, a further threshold value for an inertial angle, for an inertial angle velocity, for a ground reaction force, for a joint torque, for a joint angle or for a distance of a force vector from a joint component is not reached within a fixed time.

37. The method as claimed in claim 35 or 36, characterized in that the resistance remains reduced if a joint angle increase is detected.

38. The method as claimed in one of the preceding claims, characterized in that the point at which a force acts on the foot is determined and the resistance is increased, or not reduced, if the point at which a force acts moves in the direction of the heel.

39. The method as claimed in one of the preceding claims, characterized in that the bending resistance is increased, or not reduced, in the standing phase if an inertial angle of a lower leg part that is decreasing in the direction of the vertical and simultaneously a loading of the forefoot are determined.

40. The method as claimed in claim 39, characterized in that the resistance is increased, or not reduced, if the inertial angle velocity of a joint part falls below a threshold value.

41. The method as claimed in claim 39 or 40, characterized in that the variation in the loading of the forefoot is determined and the resistance is increased, or not reduced, if, with a decreasing inertial angle of the lower leg part, the loading of the forefoot is reduced.

42. The method as claimed in one of claims 39 to 41, characterized in that a knee torque is detected and the resistance is increased, or not reduced, if a knee torque acting in the direction of flexion is determined.

43. The method as claimed in one of claims 39 to 42, characterized in that the inertial angle of the lower leg part is determined either directly or from the inertial angle of another connection part and a joint angle.

44. The method as claimed in one of claims 39 to 43, characterized in that a changing of the inertial angle of a joint part is determined directly by way of a gyroscope or from the differentiation of an inertial angle signal of the joint part or from the inertial angle signal of a connection part and a joint angle.
